SEQUENCE LISTING (2 sequences)

SEQ ID NO: 1 - protein, 8 aa, Artificial Sequence
  Synthetic Construct
EEEDDDDE

SEQ ID NO: 2 - protein, 10 aa, Artificial Sequence
  Synthetic Construct
LKEKYEKDIA

(19) United States
(12) Patent Application Publication
(10) Pub. No.: US 2010/0172909 A1
(43) Pub. Date: Jul. 8, 2010

(54) Cerebral edema suppressant

(76) Inventors: Masahiro Nishibori, Okayama-shi (JP); Shuji Mori, Okayama-shi (JP); Hideo Takahashi, Okayama-shi (JP); Yasuko Tomono, Okayama-shi (JP); Naoto Adachi, Kyoto-shi (JP); Keyue Liu, Touon-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/654,790

(22) Filed: Jan. 4, 2010

Related U.S. Application Data
(63) Continuation-in-part of application No. 12/084,044, filed on Apr. 24, 2008, now abandoned.

(30) Foreign Application Priority Data
Oct. 24, 2005 (JP) 2005-308949

(51) Int. Cl. A61K 39/395; A61P 43/00

(52) U.S. Cl. 424/139.1

(57) ABSTRACT

The objective to be solved by the present invention is to provide a method for effectively suppressing cerebral edema. The method for suppressing cerebral edema according to the present invention is characterized in comprising a step of administering an anti-HMGB 1 antibody recognizing 208EEEDDDDE215 (SEQ ID NO:1) as an HMGB1 epitope.

### TECHNICAL FIELD

The present invention relates to a drug for suppressing cerebral edema.

### BACKGROUND ART

Cerebral edema is a condition characterized by an excess of watery fluid collected in the extracellular spaces of the brain, such as circumferences of brain cell and cerebral blood vessel. Cerebral edema needs aggressive medical treatment, since cerebral edema increases pressure in the brain to cause death.

Cerebral edema is classified into vasogenic edema and cytotoxic edema. Vasogenic edema is caused by breakdown of blood-brain barrier due to cerebral contusion, intracerebral hemorrhage, cerebral tumor and the like, and by excessive increase of vascular permeability, which leads leakage and accumulation of plasma component outside of brain cell. Cytotoxic edema is caused by increasing fluid component in brain cell due to hypoxia, impaired metabolism and the like. It is said that the breakdown of the blood-brain barrier is not observed for the case of cytotoxic edema. However, it is difficult to clearly distinguish cytotoxic edema from vasogenic edema, since disease condition may transit from vasogenic edema to cytotoxic edema. Therefore, it is very important for suppressing cerebral edema to inhibit the breakdown of the blood-brain barrier, that is, excessive increase of vascular permeability.

As the therapeutic agent for cerebral edema, glycerol, mannitol, diuretics, and the like are exemplified. However, a more effective therapeutic agent is required.

HMGB1, i.e. High Mobility Group box 1, is a protein in which 95% or more of amino acid sequence is equal from a rodent to a human. The HMGB1 is present in a normal cell. However, the blood concentration thereof is increased by stimulation with LPS (liposaccharide) which is an endotoxin released in sepsis, one of systemic inflammatory response syndromes, leading to final tissue failure. Therefore, in the technology described in Published Japanese Translation of PCT International Publication No. 2003-520763, an HMG antagonist is administered in order to treat a symptom having a characteristic of activation of an inflammatory cytokine cascade. However, there is neither description nor suggestion about not only cerebral edema but also general edema in the publication. In addition, it is described in Published Japanese Translation of PCT International Publication No. 2005-512507 that the anti-HMGB1 antibody may treat a disease associated with an inflammatory cytokine cascade. However, the anti-HMGB1 antibody specifically binds to the box part of HMGB1, and there is neither description nor suggestion about not only cerebral edema but also general edema in the publication.

In addition, it is described in Published Japanese Translation of PCT International Publication No. 2005-537253 that an HMGB1 antagonist such as anti-HMGB1 antibody is used for treating edema and the like. However, the anti-HMGB1 antibody actually used in the publication is antibodies which specifically bind to box or residues 166 to 181. Concerning the anti-HMGB1 antibody, it is only experimentally demonstrated that certain anti-HMGB1 antibody inhibits cell migration of HMGB1, and it is not experimentally demonstrated at all that the anti-HMGB1 antibody is useful for suppressing edema. Cerebral edema is not described in the publication either.

### DISCLOSURE OF THE INVENTION

Under the above circumstance, the objective to be solved by the present invention is to provide a method for effectively suppressing cerebral edema.

The present inventors have continuously studied anti-HMGB1 antibody. As a result, the present inventors found out that the anti-HMGB1 antibody which specifically binds to C-tail of HMGB1 can effectively suppress cerebral edema, resulting in completion of the present invention.

The method for suppressing cerebral edema according to the present invention is characterized in comprising a step of administering an anti-HMGB1 monoclonal antibody recognizing 208EEEDDDDE215 as an HMGB1 epitope.

According to the present invention method, cerebral edema can be effectively suppressed. In addition, it is considered that a possibility generating a serious adverse side effect is extremely low based on the relatively safe clinical application of antibody drugs currently used Therefore, the method for suppressing cerebral edema according to the present invention is extremely useful.

### BRIEF DESCRIPTION OF THE DRAWINGS

FIG. 1 shows the effects of the anti-HMGB1 monoclonal antibody which specifically binds to C-tail with neutralizing the activity of HMGB1. FIG. 1(A) shows the levels of ICAM-1 expression on monocytes by addition of HMGB1, and FIG. 1(B) shows the levels of ICAM-1 expression in case that the anti-HMGB1 monoclonal antibody is added simultaneously with addition of HMGB1.

FIG. 2 shows the suppressing effects of administration of the anti-HMGB1 monoclonal antibody which specifically binds to C-tail on the increased permeability of brain blood vessel. In non-ischemic group without load of cerebral ischemia, leakage of Evans blue, i.e. dye, is not seen. On the other hand, in a control antibody-administered group to which cerebral ischemia was loaded and a control antibody was administered, a considerable amount of leakage is observed. In an anti-HMGB1 antibody-administered group to which the anti-HMGB1 monoclonal antibody of the present invention was administered, such a leakage is clearly inhibited.

FIG. 3 summarizes the quantitative comparison of the leakage of Evans blue into brain tissue between the control antibody-administered group and the anti-HMGB1 antibody-administered group. It is found that the increased permeability of brain blood vessels can be significantly inhibited by administering the anti-HMGB1 monoclonal antibody.

FIG. 4 is a graph showing the relative amount of dye (Evans blue) which extravasated from microvasculature after administration of saline, HMGB1 only, HMGB1+the anti-HMGB1 monoclonal antibody according to the present invention, or HMGB1+a monoclonal antibody binding to box of HMGB1, to the dorsal skin of experimental rats.

### BEST MODE FOR CARRYING OUT THE INVENTION

In the present invention method, the cerebral edema suppressant which contains the anti-HMGB1 monoclonal antibody specifically binding to C-tail of HMGB1 as active ingredients is administered.

The anti-HMGB1 monoclonal antibody according to the present invention specifically binds to C-tail of HMGB1 and neutralizes HMGB1, and inhibits the increased permeability of brain blood vessel, to suppress cerebral edema. On the other hand, the antibody does not act on the other parts of HMGB1 and other substances. Therefore, it is considered that there is no or extremely little possibility of production of an adverse side effect.

The anti-HMGB1 monoclonal antibody may be prepared according to a conventional method. For example, a mouse, a rat or the like is immunized using commercially available HMGB1, and its antibody-producing cell or spleen cell and a myeloma cell are fused to obtain a hybridoma. The hybridoma is cloned, and a clone producing an antibody which specifically reacts with HMGB1 is screened. The clone is cultured, and the antibody which specifically binds to C-tail of HMGB1 is purified among the secreted monoclonal antibody.

The kind of the anti-HMGB1 monoclonal antibody used in the present invention is not specifically limited. For example, a humanized antibody and a complete human antibody may be used. The antibody which is derived from an object animal to be administered is preferably used.

A dosage form of the cerebral edema suppressant according to the present invention is not specified. However, liquid preparations such as a solution and an emulsion preparation are preferable for the administration as injection, taking into consideration the fact that the anti-HMGB1 monoclonal antibody as an active ingredient is a peptide.

A solution isotonic to plasma, such as a pH-adjusted physiological saline and an aqueous solution of glucose can be used as a solvent for a liquid preparation. When the antibody is freeze-dried together with a salt or the like, pure water, distilled water, sterilized water and the like can also be used. The concentration may be that of a common antibody preparation; and may generally be about 1 mg/mL or more and 5 mg/mL or less. However, the osmotic pressure of the injection needs to be similar to that of plasma.

In the present invention, “suppression” implies both concepts of the inhibition of cerebral edema, i.e. “prevention”, and the relief or the inhibiting development of occurred cerebral edema, i.e. “treatment”. Consequently, in the present invention method, the suppressant for cerebral edema may be administered for the preventive purpose before the formation of cerebral edema, or for the treatment purpose after the formation of cerebral edema.

As shown in the Examples described later, prominent suppressing effects on the increased permeability of brain blood vessel were observed in the case where 200 μg of the anti-HMGB1 monoclonal antibody was administered. From the result, dose of the anti-HMGB1 monoclonal antibody for humans is estimated to be 0.2 to 5 mg/kg, preferably 0.2 to 2 mg/kg per dosage. However, the dose of the suppressant should be suitably changed depending on patient's age, sex and severity of illness and the like.

In the present invention method, the cerebral edema suppressant may be administered by intravenous injection. It is at least experimentally demonstrated that the cerebral edema suppressant according to the present invention can suppress cerebral edema by intravenous injection, though it is unknown whether the suppressant acts on brain blood vessel to inhibit the increased permeability or acts on brain cell through the blood-brain barrier.

Cerebral edema suppressant results from a number of causes. It is preferable to administer the cerebral edema suppressant according to the present invention immediately after the cause of cerebral edema arises, since it becomes possible the suppressant can prevent plasma proteins from extravasating from brain blood vessel and can prevent the occurrence of cerebral edema even after the cause of cerebral edema arises. The term, “immediately after”, in the present invention preferably means not more than 5 hours, more preferably not more than 2 hours, even more preferably not more than 1 hour.

### Examples

The present invention will be explained more specifically by examples below. However, the present invention is not limited by the following examples, and various alterations can be made on it to an extent applicable to the above-described and later-described points. All of them are included in the technical scope of the present invention.

(a) Immunization of Rat

Into a 2 mL-glass syringe, was taken 1 mg/mL of a commercially available mixture of bovine thymus-derived HMGB1 and HMGB2 (manufactured by Wako Pure Chemical Industries, Ltd., code No. 080-070741), and an equivalent volume of a complete Freund's adjuvant was taken into another 2 mL-glass syringe. These syringes were connected with a connecting tube. The mixture and the adjuvant were gradually kneaded through the connecting tube to obtain an emulsion. Each 0.1 mL of the obtained emulsion at a total of 0.2 mL was injected to a rat anesthetized with sevoflurane in a hind limb footpads. After 2 weeks, blood was probatively taken from jugular, and the increase of antibody titer was confirmed. Then, an enlarged iliac lymph node was sterilely taken out 5 weeks after the injection administration. From the two lymph nodes obtained, about 6×10⁷ cells could be recovered.

(b) Cell Fusion and Cloning

The iliac lymph node cell and mouse myeloma SP2/O-Ag14 (SP2) cell were fused using polyethylene glycol, and the obtained fused cell was seeded on a 96-well microplate. After one week, initial ELISA screening was performed, and positive wells were subjected to secondary screening by Western blotting. Well cells exhibiting positive were transferred to a 24-well microplate, and the cells were increased to about 2×10⁵ as the almost confluent state. Then, using 0.5 mL of a freezing medium in which 10% bovine fetal serum and 10% dimethyl sulfoxide were added to a GIT medium, the cells were freezing-stored in liquid nitrogen. The freezing-stored cells were thawed, and then subjected to cloning on a 96-well microplate.

(c) Purification of Antibody

The positive cells were cultured for 2 weeks at a large scale with a rotation culturing device (manufactured by Vivascience) to obtain an antibody fluid having a concentration of 2 to 3 mg/mL. The antibody fluid was kneaded with an affinity gel (manufactured by Invitrogen, MEP-HyperCel) under neutral pH to specifically bind the anti-HMGB1 antibody to the gel. The antibody which specifically bound to the gel was eluted by a glycine-hydrochloric acid buffer at pH of 4. The eluate was concentrated with an ultra filtration devise, and thereafter the antibody was further purified with a Sepharose CL6B gel filtration column of diameter 2 cm×length 97 cm.

The one antibody among the obtained monoclonal antibodies specifically recognizes an amino acid sequence in C-tail of HMGB1, 208EEEDDDDE215, in which E stands for glutamic acid and D stands for aspartic acid, as an epitope . Though HMGB2 is a protein similar to HMGB1, HMGB2 lacks the sequence: DDDDE after 211; therefore, the monoclonal antibody of the present invention does not bind to HMGB2, and can specifically recognize and bind to only HMGB1.

### Comparative Example 1

### Preparation of the anti-HMGB1 Monoclonal Antibody Binding to B-box of HMGB1

The anti-HMGB1 monoclonal antibody which recognizes B-box of HMGB1, of which sequence is LKEKYEKDIA, as an epitope was isolated and purified in a similar method of the above Example 1.

### Example 2

### Determination of the Binding Site of the Antibodies

The binding site of the monoclonal antibodies prepared in the above Example 1 and Comparative Example 1 was determinated once again.

Specifically, 41 peptides having 15 amino acid sequences such as 1 to 15, 6 to 20, 11 to 25 - - - (as the case may be) from the N-terminus among the amino acid sequence of HMGB1 were synthesized. The 1 mg/mL solutions of the peptides were added dropwise onto Ultra Bind Membrane, manufactured by PALL Life Science, by 0.5 μL each to eliminate overlap with each other. Further, 1 mg/mL solution of purified HMG-1,2 derived from bovine thymus was also added dropwise by 0.5 μL as a positive control. After the dropwise addition, the membrane was air-dried under room temperature for 1 hour. After air drying, the membrane was blocked with a 20% skim milk/PBS solution for 1hour. The membrane was then washed two times with PBS for 5 minutes, and air-dried under room temperature for 1 hour. The membrane was soaked into PBS, and each antibody solution (1 mL) diluted 500-fold with a 1% BSA/PBS solution was put into a nylon membrane and packed and was allowed to react at 4° C. overnight. The membrane was then washed three times with a 0.1% Tween 20/PBS solution for 5 minutes. An anti-RAT antibody labeled with peroxidase was diluted 1000-fold with a 1% BSA/PBS solution, and the solution (1 mL) was added thereto and was allowed to react at room temperature overnight. The membrane was then washed three times with a 0.1% Tween 20/PBS solution for 5 minutes, and was color-developed by a chemiluminescence method.

It could be confirmed that the antibody obtained in the above Example 1 specifically binds to C-tail of HMGB1, of which sequence is EEEDDDDE, and the antibody obtained in the above Comparative Example 1 specifically binds to B-box of HMGB1, of which sequence is LKEKYEKDIA, from the amino acid sequence of which color appeared.

### Example 3

The neutralization activity of the anti-HMGB1 monoclonal antibody prepared in Example 1 was tested.

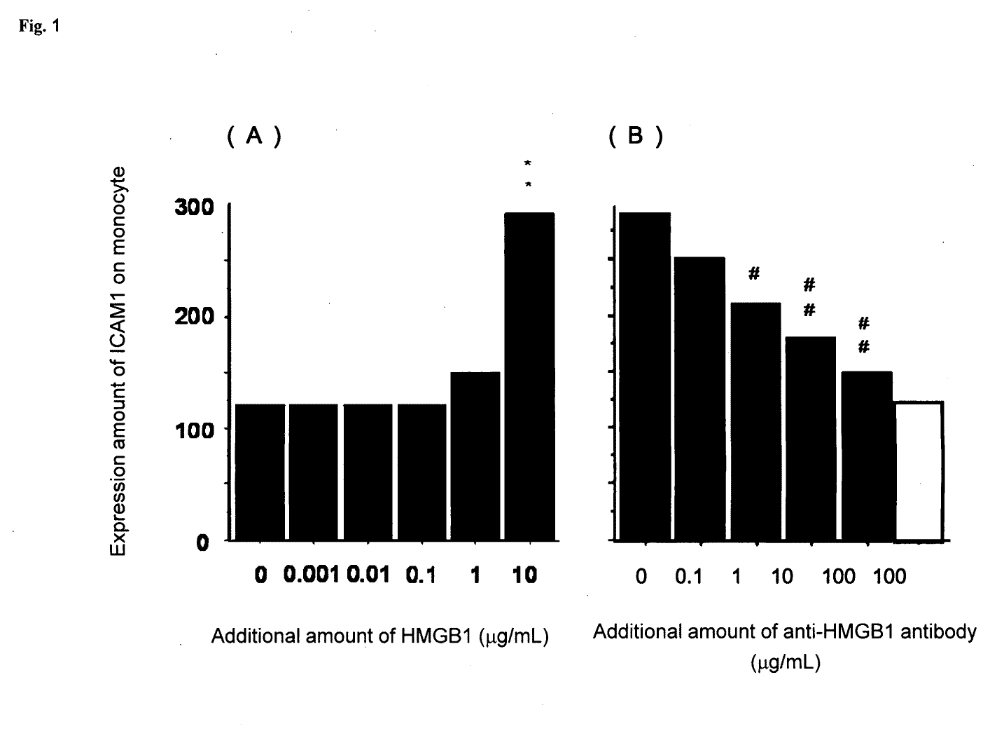

First, 1×10⁶/mL peripheral blood mononuclear cells were prepared from peripheral blood of a healthy person by a conventional method, and were cultured for 24 hours in a basal medium for culturing an animal cell containing 10% bovine fetal serum (manufactured by Sigma, RPMI1640). Then, bovine HMGB1 purified from a bovine thymus-derived HMGB1/2 mixture manufactured by Wako Pure Chemical Industries, Ltd. was added to the medium at a concentration of 0.001 to 10 μg/mL to stimulate the monocytes. Twenty four hours after addition of HMGB1, the cells were collected, and an expression amount of ICAM-1 (intercellular adhesion molecule-1) expressed on the monocytes with HMGB1 was quantitated by a fluorescent antibody method (FACS method). Results are shown in FIG. 1(A). In FIG. 1(A), “**” indicates the case where there was a significant difference at p<0.01 by t-test as compared with the case of addition of no HMGB1. From the result, it was found that expression of ICAM-1 on monocytes is significantly increased by 10 μg/mL of HMGB1.

Then, in the above procedure, 0 to 100 μg/mL of anti-HMGB1 monoclonal antibody was added at the same time with the addition of 10 μg/mL of HMGB1, and expression levels of ICAM-1 were quantitated using a fluorescent antibody method as described above. Results are shown in FIG. 1(B). In FIG. 1(B), “#” indicates the case where there was a significant difference at p<0.05 by t-test as compared with the case of addition of no antibody, and “##” indicates the case where there was a significant difference at p<0.01. In addition, a rightmost outline column is the result of the case of addition of no HMGB1. From the result, it was verified that the anti-HMGB1 monoclonal antibody prepared in Example 1 at a concentration of 1 μg/mL or higher could significantly neutralize HMGB1.

### Example 4

### Vascular Permeability Test

Nine male Wistar rats were divided into an anti-HMGB1 monoclonal antibody-administered group of 3 animals, a control antibody-administered group of 3 animals and a non-administered group of 3 animals. These rats were anesthetized with a gas mixture of 2% halothane and 50% laughing gas, and kept under spontaneous breathing. Subsequently, a median incision was made in the neck of the rat placed on its back, and the right common carotid artery was exposed. After an intraperitoneal injection of 100 units of heparin, the root of the right middle cerebral artery was occluded by inserting 4.0 nylon thread coated with silicone into the right internal carotid artery from the bifurcation of the internal and external carotid arteries. The tip of the nylon thread was placed 18 mm from the bifurcation. After suture of the incision of the skin, the rats were allowed to recover from anesthesia. During surgery, an electronic thermometer was inserted into the rectum, and the rectum temperature was maintained at 37.0±0.1° C. with a lamp. After recovery from anesthesia, paralysis of the contralateral limb was observed in all rats.

Five minutes before reperfusion of blood flow, the rats were anesthetized again. After opening the skin suture, cerebral blood flow was resumed by removing the nylon thread by 5 mm 2 hours after middle cerebral artery occlusion. To the anti-HBG1 monoclonal antibody-administered group, was administered 200 μg of the anti-HMGB1 monoclonal antibody of the above Example 1 immediately after blood stream recovery. Then, immediately after administration of the anti-HMGB1 monoclonal antibody, 2% Evans blue saline was administered at a dose of 20 mg/kg through a tail vein. Since Evans blue is bound to albumin which is a serum protein, albumin leaked out from a blood vessel can be visualized. To the control antibody-administered group, were administered the same amount of an anti-Keyhole Limpet monoclonal antibody and Evans blue. The antibody belongs to the same IgG2a class as that of the anti-HMGB1 monoclonal antibody. In addition, to the non-administered group, only cervical operation was applied, and no local cerebral ischemia was loaded, and only Evans blue was administered as described above.

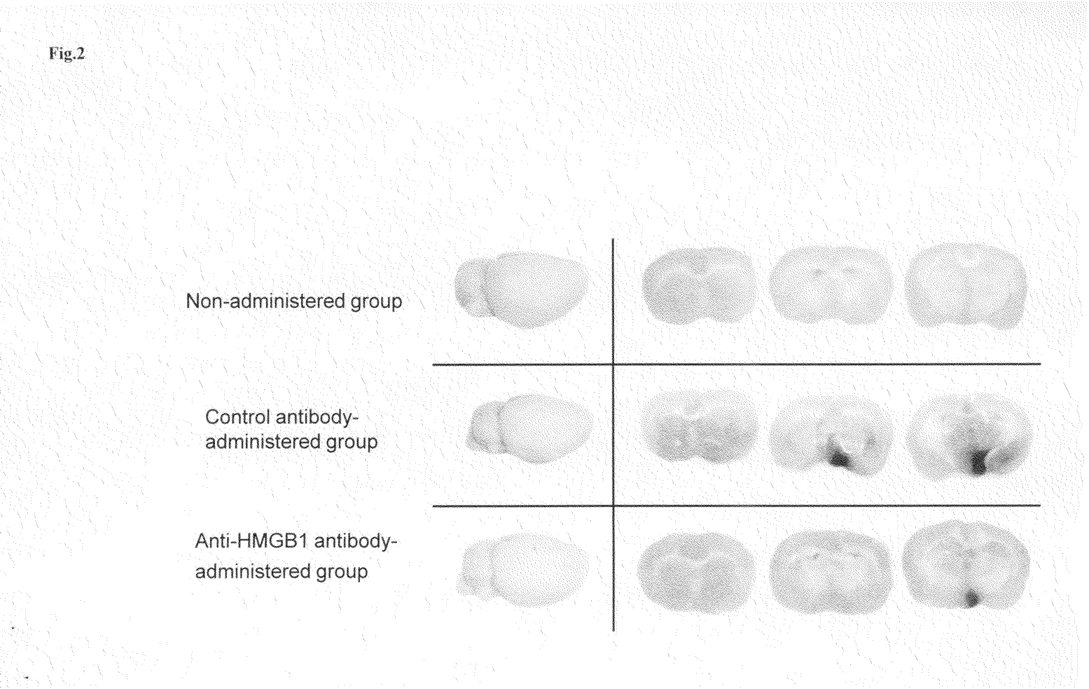

Three hours after administration of Evans blue and the like, 50 mg/kg pentobarbital was administered intraperitoneally, 150 ml of physiological saline was perfused through a left ventricle under deep anesthesia, and then a brain was isolated. FIG. 2 shows a photograph of sections of the isolated brain. In the non-administered group, transfer of Evans blue from a blood vessel to the brain is hardly recognized. In the control antibody-administered group, transfer of Evans blue into a brain is recognized in any of hypothalamus, corpus striatum and cerebral cortex on an ischemia side, indicating that brain blood vessel permeability of these regions was increased. On the other hand, the permeability of brain blood vessel observed in the control antibody-administered group was remarkably suppressed in the anti-HMGB1 antibody-administered group. Especially, leakage of Evans blue was almost suppressed in corpus striatum and cerebral cortex in which an infarction lesion is formed after 24 hours.

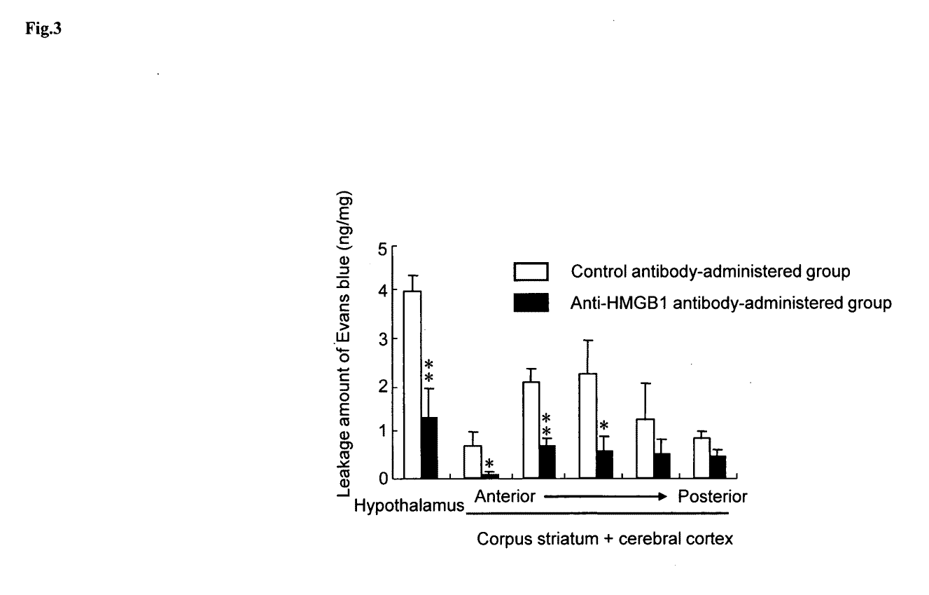

In addition, a leakage amount of Evans blue was quantitated by homogenizing the brain of the anti-HMGB1 antibody-administered group and the control antibody-administered group in a mixed solvent of 0.6 NH₃PO₄:acetone=5:13 and by extracting Evans blue. Results are shown in FIG. 3. In FIG. 3, “*” indicates the case where the value was significant at <0.05 relative to the corresponding control, and “**” indicates the case where the value was significant at p<0.01. From the result, it was verified that the anti-HMGB1 monoclonal antibody can significantly suppress leakage of Evans blue.

From the above results, it is found that, by administering the anti-HMGB1 monoclonal antibody of the above Example 1, leakage of a blood protein into brain tissue due to cerebral ischemia can be effectively suppressed, and it is possible to suppress brain edema which is a cause for a brain disorder.

### Example 5

### Vascular Permeability Test

Eight male Wistar rats weighing about 300 to 500 g were anesthetized with a gas mixture of 2% halothane, 49% oxygen and 49% laughing gas under spontaneous breathing. Subsequently, the rats were put placed on the stomach, the back fur was shaved, and ten parts with 1 cm intervals on both sides of the spine were marked. Separately, human HMGB1 recombinant was prepared by using an insect cell Sf9. Only saline (0.1 mL), only 2.5 μg/mL solution of HMGB1 (0.1 mL) , 2.5 μg/mL solution of HMGB1 (0.1 mL) and 25 μg/mL solution of the anti-HMGB1 monoclonal antibody binding to C-tail (Example 1) (0.1 mL) , or 2.5 μg/mL solution of HMGB1 (0.1 mL) and 25 μg/mL solution of the anti-HMGB1 monoclonal antibody binding to B-box (comparative example 1) (0.1 mL) was administered by subcutaneous injection into the every two marked spots on the rats respectively.

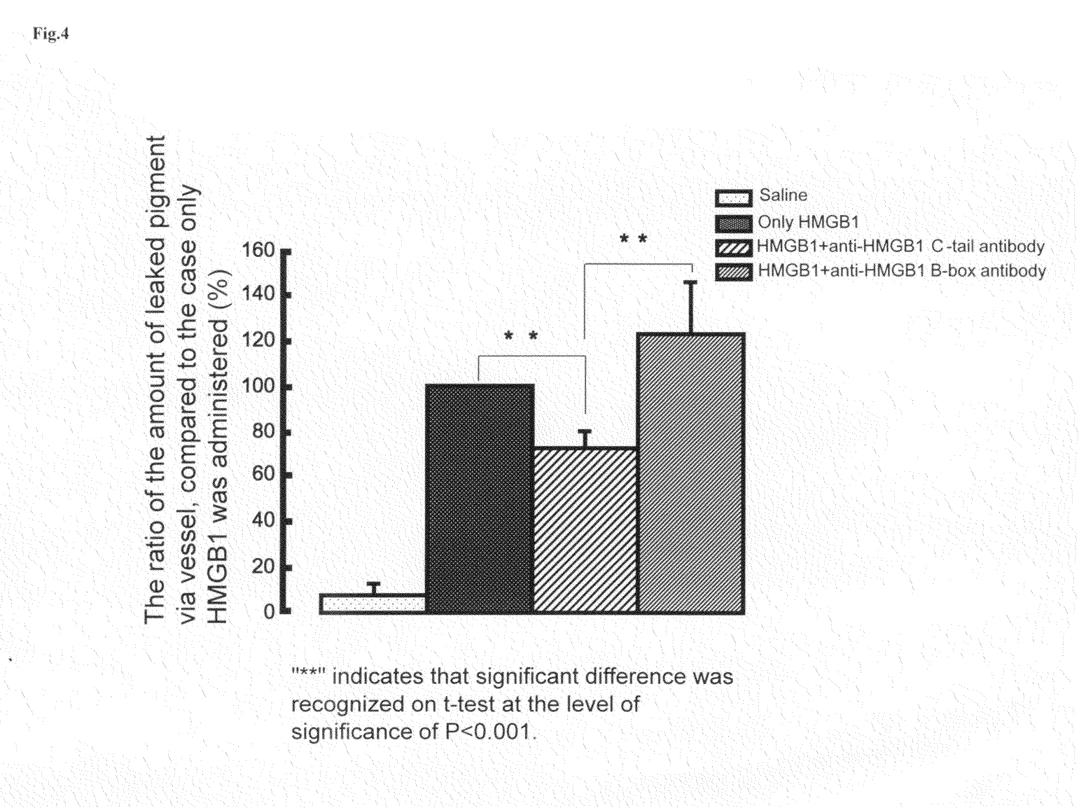

One hour after the subcutaneous injection, 2% Evans blue solution in saline was administered at the rate of 2 mL per kg weight by intravenous injection. Two hours after the intravenous injection of Evans blue, pentobarbital sodium was intraperitoneally administrated to the rats at the dose of 50 mg per kg weight so that the rats were kept in deep-anesthesia state. Saline (100 mL) was infused from the left ventricle, and the blood was removed from the right atrium. Back skin of the rats was peeled off and was photographed from the rear side. The picture was analyzed by a medical image analyzing software (Image J, NIH), to measure the concentration and area of dye which was leaked out of vessel from the blood and calculate the amount thereof. The extravascular leakage amount of the dye in case where HMGB1 was administrated alone is set as 100%, and the ratios of leakage relative to 100% are shown in FIG. 4.

As shown in FIG. 4, in case where only HMGB1 was administrated, the amount of leaked d_{y}e via vascular endothelial cells from the blood was naturally increased, compared to the case only saline was administrated. However, even in case of administrating the anti -HMGB1 monoclonal antibody binding to B-box in addition to HMGB1, the extravascular leakage amount of the dye tended to rather increase, although there was no significant difference.

On the other hand, in case of administrating the anti-HMGB1 monoclonal antibody binding to C-tail in addition to HMGB1, extravascular leakage of the dye was significantly suppressed at the level of significance of p<0.001, compared to not only the case of administrating HMGB1 alone but also the case of administrating the anti-HMGB1 monoclonal antibody binding to B-box.

As the above mentioned result, it can be thought that the anti-HMGB1 monoclonal antibody binding to B-box cannot suppress cerebral edema at all, since the antibody cannot prevent the extravascular leakage of dye, whereas it can be thought that the anti-HMGB1 antibody binding to C-tail according to the present invention can remarkably suppress cerebral edema, since the antibody can effectively prevent the extravascular leakage of dye.

1. A method for suppressing cerebral edema, comprising a step of administering an anti-HMGB1 antibody recognizing 208EEEDDDDE215 (SEQ ID NO:1) as an HMGBI epitope.

2. The method for suppressing cerebral edema according to claim 1, wherein the anti -HMGBI antibody is administered by intravenous injection.

3. The method for suppressing cerebral edema according to claim 1, wherein the anti-HMGB1 antibody is administered immediately after cause of cerebral edema arises.

4. The method for suppressing cerebral edema according to claim 3, wherein the anti-HMGB 1 antibody is administered within 5 hours after cause of cerebral edema arises.
